(19) United States
(12) Patent Application Publication
(10) Pub. No.: US 2022/0040058 A1
(43) Pub. Date: Feb. 10, 2022

(54) COSMETIC HAIR COMPOSITION IN THE FORM OF A NANOEMULSION COMPRISING A PARTICULAR NON-IONIC SURFACTANT AND A PROPELLANT

(71) Applicant: L'OREAL, Paris (FR)

(72) Inventors: Sylvia DA SILVA, Saint Ouen (FR); Ségolène DE MENTHIERE, Saint Ouen (FR); Aldo PIZZINO, Saint Ouen (FR)

(21) Appl. No.: 17/414,109

(22) PCT Filed: Dec. 20, 2019
(86) PCT No.: PCT/EP2019/086555

§ 371 Date: Jun. 15, 2021

(30) Foreign Application Priority Data
Dec. 20, 2018 (FR) 1873600

(51) Int. Cl. A61K 8/06; A61K 8/86; A61K 8/31; A61K 8/39; A61K 8/92; A61Q 5/06; A61K 8/04

(52) U.S. Cl. CPC A61K 8/062; A61K 8/86; A61K 8/31; A61K 8/39; A61K 2800/87; A61Q 5/06; A61K 8/046; A61K 2800/413; A61K 2800/591; A61K 8/922

(57) ABSTRACT

The present invention relates to a cosmetic composition in the form of a nanoemulsion comprising at least one particular non-ionic surfactant, at least one fatty substance, water and at least one propellant. The invention also relates to an aerosol device and also to a process for styling keratin fibres using such a composition.

The present invention relates to a cosmetic composition in the form of a nanoemulsion comprising at least one particular non-ionic surfactant, at least one fatty substance, water and at least one propellant.

The invention also relates to an aerosol device and also to a process for styling keratin fibres using such a composition.

Styling products are normally used to construct and structure the hairstyle and to give it shape retention. They are usually in the form of lotions, gels, mousses, creams, sprays, etc. These compositions generally comprise one or more film-forming polymers or “fixing polymers”. These polymers allow the formation of a coating film on the hair, thus providing form retention of the hairstyle.

Products which are hairstyle or hairstyle shape retention aids and which do not comprise a fixing polymer also exist.

These compositions may be in the form of hair gels, sprays or mousses which are generally applied to wet hair, which is shaped before performing blow drying or drying.

Hairstyling products in aerosol devices are generally available in an opaque container since they do not generally have a sufficiently attractive appearance, in particular in the presence of a propellant, especially in the presence of a liquefied propellant, and are generally not stable over time and/or with respect to temperature.

Moreover, an increasing number of users of hairstyling products are especially searching for compositions which are more attractive, more fluid or even clearer.

The applicant has now discovered, surprisingly, that a seemingly single-phase cosmetic composition in the form of a nanoemulsion of oil-in-water type comprising at least one particular non-ionic surfactant, at least one fatty substance having a melting point less than or equal to 35° C., one or more propellants and water makes it possible to solve the abovementioned problems.

A subject of the present invention is thus a cosmetic composition in the form of a nanoemulsion of oil-in-water type, comprising:

(i) at least one non-ionic surfactant of formula (I):

R—O—(CH₂—CH₂—O)ₙ—H (I)

in which:
R is a linear or branched C₈-C₄₀ alkenyl radical;
n is an integer ranging from 6 to 20;

(ii) one or more fatty substances having a melting point less than or equal to 35° C. at atmospheric pressure;

(iii) one or more propellants; and

(iv) water.

The composition according to the invention, when it is propelled by means of a conventional aerosol device, makes it possible in particular to dispense a uniform, firm and creamy mousse which holds well in the hand.

Furthermore, the mousse formed from the composition according to the invention spreads easily and uniformly on the hair, and has good styling properties. It especially affords good hold of the head of hair over time under both wet and dry conditions, while at the same time giving the head of hair volume, and without making the hairstyle rigid. In the case of curly hair, the composition according to the invention affords good curl definition.

In particular, the composition according to the invention gives the hairstyle a natural look and fluid movement.

It also gives the hair a particularly soft and pleasant feel.

The composition according to the invention is preferably clear, which gives it a particularly attractive aesthetic appearance that is highly sought after by users. In particular, when it is packaged in a pressurized device, for example of aerosol type, the propellant(s) are dispersed in the droplets of the nanoemulsion, which gives the composition a particularly aesthetic homogeneous appearance. The applicant has also noted that the aesthetic appearance of the composition according to the invention is particularly augmented when it is packaged in a container that is itself transparent.

It has also been noted that the composition according to the invention is stable over time and with respect to temperature. In particular, the appearance of the composition according to the invention remains significantly the same after 2 months of storage at ambient temperature (25° C.).

In addition, the composition according to the invention can be of variable viscosity, in particular it can be gelled without needing the presence of thickeners such as thickening polymers.

A subject of the present invention is also an aerosol device comprising a composition according to the invention, a container containing said composition, and a means for spraying said composition.

A subject of the invention is also a process for styling keratin fibres, in particular human keratin fibres such as the hair, comprising the application to said fibres of the composition, this application being optionally followed by rinsing after an optional leave-on-time.

Other subjects, characteristics, aspects and advantages of the invention will emerge even more clearly on reading the description and the example that follows.

In the present description, and unless otherwise indicated:

the expression “at least one” is equivalent to the expression “one or more” and can be substituted for said expression; the expression “between . . . and . . .” is equivalent to the expression “ranging from . . . to . . .” and can be substituted for said expression, and implies that the limits are included.

According to the present application, the term “keratin fibres” denotes human keratin fibres and more particularly the hair.

For the purposes of the present invention, the term “polyoxyalkylenated compound” is intended to mean a compound comprising at least one polyoxyethylene and/or polyoxypropylene and/or polyglycerol group; preferably, the number of ethylene oxide and/or propylene oxide groups possibly ranging from 1 to 100, and the number of glycerol groups possibly ranging from 2 to 30.

For the purposes of the present invention, the term “non-polyoxyalkylenated compound” is intended to mean a compound not comprising several polyoxyethylene and/or polyoxypropylene and/or polyglycerol groups.

The composition according to the invention is in the form of an oil-in-water nanoemulsion, the particles of which preferably have a number-average size of less than 200 nm, preferentially of between 10 and 150 nm, and better still between 10 and 100 nm, and even better still between 10 and 90 nm, or even between 10 and 80 nm, even better still between 10 and 75 nm.

The number-average size of the particles (or oil drops) can be determined in particular according to the known Dynamic Light Scattering (DLS) method. By way of apparatus that can be used for this determination, mention may be made of the Zetasizer Nano ZS granulometer from Malvern Instrument. This granulometer is equipped with a standard laser (Helium-Neon) of 4 mW of power at a wavelength λ of 633 nm. In particular, the nanoemulsion is previously diluted with distilled water with a dilution ratio of 1/100, and the temperature of the sample is regulated at 25° C.

In addition, the composition according to the invention has a very low polydispersity, that is to say that the size of the particles (of the objects) is very homogeneous. The particles present in the composition according to the invention are droplets of oily phase containing the propellant(s), within the dispersant aqueous phase.

The combination according to the invention has the advantage of resulting in the formation of a composition in the form of a nanoemulsion of oil-in-water type starting from any type of preparation process, for example by means of a high-pressure homogenization process, a phase inversion (PIT) process or a dilution process, and in particular starting from a low-energy process that can be easily industrialized.

In particular, such a combination allows the formation of a composition in the form of a nanoemulsion of oil-in-water type starting from a phase inversion (PIT) process, in conventionally industrial equipment, in particular jacketed tanks, which are significantly less expensive to implement than a high-pressure homogenization process, in particular in terms of purchase, energy and maintenance.

The non-ionic surfactants of formula (I) according to the invention are in particular easy to use industrially, thereby making them more manipulable during the process for preparing the composition according to the invention.

In other words, the composition according to the invention can be obtained by means of any type of process, in particular by means of a phase inversion (PIT) process.

Preferably, the cosmetic composition according to the present invention is seemingly single-phase. For the purposes of the present invention, the term “seemingly single-phase” is intended to mean that the composition according to the invention is constituted, at ambient temperature)(25°) and atmospheric pressure, of two or more phases, where one of the phases is dispersed in the other, such that it is not possible to distinguish the phases from one another with the naked eye.

The composition according to the invention is advantageously in the form of a clear to translucent, more preferentially clear, fluid.

The clarity of the composition according to the invention can be characterized by the measurement of its turbidity, by turbidimetry (in NTU units). In the context of the present invention, the turbidity measurements were carried out with a turbidimeter, model HI 88713-ISO from the company Hanna Instruments.

Preferably, the turbidity of the compositions according to the invention, measured at ambient temperature (25° C.) and atmospheric pressure, is less than 400 NTU units, more preferentially between 1 and 250 NTU units, even better still between 3 and 200 NTU units.

Non-ionic Surfactants of Formula (I)

The composition according to the present invention comprises at least one non-ionic surfactant of formula (I):

R—O—(CH₂—CH₂—O)ₙ—H (I)

in which:
R is a linear or branched C₈-C₄₀ alkenyl radical;
n is an integer ranging from 6 to 20.

Preferably, R represents a linear or branched C₁₂-C₃₀more preferentially C₁₆-C₂₀ alkenyl radical.

Preferably, n represents an integer ranging from 8 to 12,

According to one preferred embodiment of the invention, R represents a linear or branched C₁₆-C₂₀ alkenyl radical; and/or n represents an integer ranging from 8 to 12.

According to another preferred embodiment of the invention, the composition comprises at least one non-ionic surfactant of formula (I), chosen from oleyl alcohol comprising 8 mol of ethylene oxide, oleyl alcohol comprising 10 mol of ethylene oxide, and oleyl alcohol comprising 12 mol of ethylene oxide.

According to one particularly preferred embodiment of the invention, the composition comprises oleyl alcohol comprising 10 mol of ethylene oxide (INCI name: Oleth-10).

Preferably, the total content of the non-ionic surfactant(s) of formula (I) present in the composition according to the invention is between 0.1% and 15% by weight, more preferentially between 1% and 10% by weight, even more preferentially between 2% and 8% by weight, better still between 2.5% and 5% by weight, relative to the total weight of the composition.

Fatty Substances having a Melting Point of Less Than or Equal to 35° C.

The composition according to the present invention comprises at least one fatty substance having a melting point of less than or equal to 35° C. at atmospheric pressure (1.013×10⁵ Pa).

Preferably, the fatty substances used in the composition have a melting point of strictly less than 35° C., at atmospheric pressure.

Even more preferentially, the fatty substances are liquid at 28° C. and at atmospheric pressure (1.013×10⁵ Pa).

According to one particularly preferred embodiment, the fatty substances used in the composition according to the invention are liquid at ambient temperature, that is to say that they have a melting point of less than or equal to 25° C. at atmospheric pressure (1.013×10⁵ Pa).

Preferably, the composition according to the present invention comprises at least two fatty substances having a melting point of less than or equal to 35° C. at atmospheric pressure (1.013×10⁵ Pa), preferentially at least two substances that are liquid at 28° C. at atmospheric pressure (1.013×10⁵ Pa).

The term “fatty substance” is intended to mean an organic compound which is water-insoluble at ambient temperature (25° C.) and at atmospheric pressure (1.013×10⁵ Pa), that is to say that has a solubility of less than 5% by weight, preferably less than 1% by weight, and more preferentially less than 0.1% by weight, in water. They generally have in their structure at least one hydrocarbon-based chain comprising at least six carbon atoms. They are generally soluble, under the same temperature and pressure conditions, in organic solvents such as chloroform, ethanol, benzene, liquid petroleum jelly or decamethylcyclopentasiloxane.

Preferably, the fatty substance(s) having a melting point of less than or equal to 35° C. at atmospheric pressure (1.013×10⁵ Pa), that are more preferentially liquid at 28° C. at atmospheric pressure, of the composition according to the invention are non-silicone.

The term “non-silicone fatty substance” is intended to mean a fatty substance of which the structure does not comprise a silicon atom, thus not comprising in particular a siloxane group. They generally have in their structure a hydrocarbon-based chain comprising at least six carbon atoms. Advantageously, they are not oxyalkylenated and do not contain a —COOH function.

The fatty substances that can be used in the composition according to the invention can in particular be chosen from hydrocarbons, fatty esters, fatty acids and fatty alcohols which are non-polyoxyalkylenated, these compounds being liquid at 35° C., preferentially at 28° C., at atmospheric pressure, and mixtures of these compounds.

According to the present application, the terms “fatty ester”, “fatty alcohol” and “fatty acid” denote respectively linear or branched, saturated or unsaturated, esters, alcohols and acids comprising at least one hydrocarbon-based chain containing at least six carbon atoms.

For the purposes of the present invention, the term “hydrocarbon” is intended to mean a compound comprising only carbon and hydrogen atoms.

More particularly, the hydrocarbons having a melting point of less than or equal to 35° C. at atmospheric pressure (1.013×10⁵ Pa), which are preferably liquid at 28° C., are chosen from:

linear or branched, optionally cyclic, C₆-C₁₆ alkanes. By way of examples, mention may be made of hexane, undecane, dodecane, tridecane and isoparaffins such as isohexadecane, isododecane and isodecane;

linear or branched hydrocarbons of mineral, animal or synthetic origin, having more than 16 carbon atoms, such as liquid paraffins, and derivatives thereof, petroleum jelly, liquid petroleum jelly, polydecenes, hydrogenated polyisobutene such as that sold under the brand name Parleam® by the company NOF Corporation, squalane.

Preferably, the hydrocarbon(s) are chosen from liquid paraffins, isoparaffins, liquid petroleum jelly, undecane, tridecane, isododecane and mixtures thereof.

In one most particularly preferred variant, the hydrocarbon(s) are chosen from liquid petroleum jelly, isoparaffins, isododecane and a mixture of undecane and tridecane.

For the purposes of the present invention, the term “fatty ester” is intended to mean an ester derived from a fatty acid and/or from a fatty alcohol.

More particularly, the esters having a melting point of less than or equal to 35° C. at atmospheric pressure (1.013×10⁵ Pa), which are preferably liquid at 28° C., are chosen from esters of linear or branched, saturated or unsaturated aliphatic C₁-C₂₆ mono- or polyacids which are optionally hydroxylated, and of linear or branched, saturated or unsaturated aliphatic C₁-C₂₆ mono- or polyalcohols, the total number of carbon atoms of the esters being greater than or equal to 10.

Preferably, for the esters of monoalcohols, at least one of the alcohol or the acid from which the esters of the invention are derived is branched.

Among the monoesters of monoacids and of monoalcohols, mention may be made of alkyl, in particular C₁-C₁₈ alkyl, palmitates, in particular ethyl palmitates and isopropyl palmitates, alkyl, in particular C₁-C₁₈ alkyl, myristates, such as isopropyl myristate or ethyl myristate, alkyl, in particular C₁-C₁₈ alkyl, stearates, in particular isocetyl stearate, 2-ethylhexyl isononanoate, isodecyl neopentanoate and isostearyl neopentanoate.

Use may also be made of the esters of optionally hydroxylated, C₃-C₂₂ di- or tricarboxylic acids and of C₁-C₂₂ alcohols, and the esters of optionally hydroxylated mono-, di- or tricarboxylic acids and of di-, tri-, tetra- or pentahydroxylated non-sugar alcohols which are C₄-C_{26.}

Mention may in particular be made of diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisotearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, polyethylene glycol distearates, and alkyl malates, especially (C₆-C₁₈)alkyl malates, in particular bis(C₁₂-C₁₃)alkyl malate.

Among the esters mentioned above, use is preferentially made of ethyl palmitate, isopropyl palmitate, myristyl palmitate, cetyl palmitate, stearyl palmitate, 2-ethylhexyl palmitate, 2-octyldecyl palmitate, alkyl myristates, such as isopropyl myristate, butyl myristate, cetyl myristate or 2-octyldodecyl myristate, hexyl stearate, propylene glycol dicaprylate, butyl stearate, isobutyl stearate; dioctyl malate, hexyl laurate, 2-hexyldecyl laurate, isononyl isononanoate, cetyl octanoate, and bis(C₁₂-C₁₃)alkyl malate. Among the liquid fatty esters, use may be made of esters and diesters of sugars and of C₆-C₃₀, preferably C₁₂-C₂₂, fatty acids.

The term “sugar” is intended to mean oxygen-bearing hydrocarbon-based compounds which have several alcohol functions, with or without an aldehyde or ketone function, and which comprise at least four carbon atoms. These sugars may be monosaccharides, oligosaccharides or polysaccharides.

Preferably, said sugars are chosen from sucrose, glucose, galactose, ribose, fucose, maltose, fructose, mannose, arabinose, xylose, lactose, and derivatives thereof, in particular alkylated derivatives, such as methylated derivatives, for instance methylglucose.

The esters of sugars and of fatty acids may be chosen in particular from the group comprising esters or mixtures of esters of sugars described above and of linear or branched, saturated or unsaturated C₆-C₃₀, preferably C₁₂-C₂₂, fatty acids.

If they are unsaturated, these compounds can comprise one to three conjugated or non-conjugated carbon-carbon double bonds.

The esters according to this variant can also be chosen from mono-, di-, tri- and tetraesters, polyesters, and mixtures thereof.

These esters can for example be oleates, laurates, palmitates, myristates, behenates, cocoates, stearates, linoleates, linolenates, caprates, arachidonates, and mixtures thereof, such as in particular mixed oleo-palmitate, oleo-stearate or palmito-stearate esters.

More particularly, use is made of the mono- and diesters and in particular the mono- or dioleates, stearates, behenates, oleopalmitates, linoleates, linolenates, or oleostearates of sucrose, of glucose or of methylglucose or else methylglucose dioleate (Glucate® DO).

Among the sugar esters, use may be made of pentaerythrityl esters, preferably pentaerythrityl tetraisostearate, pentaerythrityl tetraoctanoate, hexaesters of caprylic and capric acids as a mixture with dipentaerythritol.

Among the natural or synthetic esters of mono-, di- or triacids with glycerol, use may be made of vegetable or synthetic oils.

More particularly, said vegetable or synthetic oil(s) are chosen from triglyceride oils of vegetable or synthetic origin, such as liquid triglycerides of fatty acids comprising from 6 to 30 carbon atoms, for instance heptanoic or octanoic acid triglycerides or else, for example, sesame oil, soybean oil, coffee oil, safflower oil, borage oil, sunflower oil, olive oil, apricot kernel oil, camelia oil, Bambara bean oil, avocado oil, mango oil, rice bran oil, cotton seed oil, rose oil, kiwi seed oil, sea-buckthorn pulp oil, blueberry seed oil, poppy seed oil, orange seed oil, sweet almond oil, palm oil, coconut oil, vernonia oil, marjoram oil, baobab oil, rapeseed oil, ximenia oil, pracaxi oil, caprylic/capric acid triglycerides such as those sold by the company Stearineries Dubois or those sold under the names Miglyol® 810, 812 and 818 by the company Dynamit Nobel, jojoba oil, shea butter oil.

Preferably, as fatty esters having a melting point of less than or equal to 35° C. at atmospheric pressure (1.013×10⁵ Pa), that are more preferentially liquid at 28° C., use is made of triglycerides of vegetable origin, in particular coconut oil.

Particularly preferably, the composition according to the invention comprises, as fatty substances having a melting point of less than or equal to 35° C. at atmospheric pressure, that are more preferentially liquid at 28° C., at least one vegetable oil.

The term “fatty acid” is intended to mean a non-salified fatty acid, that is to say that the fatty acid must not be in the form of a generally soluble soap, that is to say that it must not be salified with a base.

More particularly, the fatty acids that can be used according to the invention are chosen from the acids of formula RCOOH, where R is a linear or branched, saturated or unsaturated radical, preferably comprising from 7 to 39 carbon atoms.

Preferably, R is a C₇-C₂₉ alkyl or C₇-C₂₉ alkenyl group, better still a C₁₂-C₂₄ alkyl or C₁₂-C₂₄ alkenyl group. R may be substituted with one or more hydroxyl groups and/or one or more carboxyl groups.

Preferentially, the fatty acid(s) having a melting point of less than or equal to 35° C. at atmospheric pressure, that are more preferentially liquid at 28° C., are chosen from oleic acid, linoleic acid and isostearic acid.

The non-polyoxyalkylenated fatty alcohols that can be used in the composition according to invention comprise from 8 to 30 carbon atoms, in particular from 10 to 24 carbon atoms, and they may be saturated or unsaturated.

The saturated non-polyoxyalkylenated fatty alcohols are preferably branched. They may or may not optionally comprise in their structure at least one aromatic ring, preferably being acyclic.

More particularly, the non-polyoxyalkylenated saturated fatty alcohols of the invention are chosen from octyldodecanol, 2-decyl-tetradecanol, isostearyl alcohol and 2-hexyldecanol.

Octyldodecanol and 2-decyltetradecanol are most particularly preferred.

The unsaturated non-polyoxyalkylenated fatty alcohols have in their structure at least one double or triple bond, and preferably one or more double bonds. When several double bonds are present, there are preferably two or three of them and they may or may not be conjugated. These unsaturated non-polyoxyalkylenated fatty alcohols may be linear or branched.

They may or may not optionally comprise in their structure at least one aromatic ring. They are preferably acyclic.

More particularly, the non-polyoxyalkylenated unsaturated fatty alcohols that can be used in the invention are chosen from oleyl alcohol, linoleyl alcohol, linolenyl alcohol and undecylenyl alcohol.

Oleyl alcohol is most particularly preferred.

Particularly preferably, the composition according to the invention comprises one or more fatty substances having a melting point of less than or equal to 35° C. at atmospheric pressure, chosen from hydrocarbons, fatty acids, non-polyoxyalkylenated fatty alcohols, fatty esters and mixtures of these compounds, in particular chosen from those mentioned above.

According to one preferred embodiment of the invention, the composition according to the invention comprises one or more fatty substances that are liquid at 28° C. at atmospheric pressure, which are more preferentially chosen from hydrocarbons, fatty acids, non-polyoxyalkylenated fatty alcohols, fatty esters and mixtures of these compounds, in particular chosen from those mentioned above.

More preferentially, the fatty substance(s) are liquid at 28° C. and at atmospheric pressure, and are chosen from hydrocarbons that are liquid at 28° C., fatty esters which are liquid at 28° C. and mixtures thereof.

Most particularly preferably, the composition according to the invention comprises one or more fatty substances having a melting point of less than or equal to 35° C. at atmospheric pressure, that are more preferentially liquid at 28° C. at atmospheric pressure, chosen from linear or branched C₆-C₁₆ alkanes; linear or branched hydrocarbons of mineral, animal or synthetic origin, having more than 16 carbon atoms; vegetable oils, in particular triglyceride oils; and a mixture of these compounds; even more preferentially chosen from undecane, tridecane, liquid paraffins and derivatives thereof, liquid petroleum jelly, polydecenes, sunflower oil, shear oil, olive oil, avocado oil, jojoba oil, soybean oil, coconut oil, rapeseed oil, and a mixture of these compounds.

According to another preferred embodiment of the invention, the composition comprises at least two fatty substances having a melting point of less than or equal to 35° C. at atmospheric pressure, that are more preferentially liquid at 28° C. and at atmospheric pressure, which belong to different families of fatty substances; and even more preferentially one of said fatty substances is chosen form hydrocarbons, and the other of said fatty substances is chosen from vegetable oils.

Preferably, the composition according to the invention comprises the fatty substance(s) (ii), that are more preferentially liquid at 28° C. and at atmospheric pressure, in a total content ranging from 0.1% to 15% by weight, even more preferentially from 0.5% to 10% by weight, and even better still from 1% to 6% by weight, relative to the total weight of the composition.

Preferably, the composition according to the invention presents a weight ratio of the content of non-ionic surfactant(s) of formula (I) to the content of the fatty substance(s) (ii), inclusively between 0.05 and 20, more preferably inclusively between 0.1 and 8; even more preferentially inclusively between 0.3 and 5; better still inclusively between 0.4 and 2; and even better still inclusively between 0.4 and 1.

Water

The composition according to the present invention comprises water.

Preferably, the total water content is between 20% and 97% by weight, preferably between 30% and 95%, preferentially between 50% and 93% by weight and even better still between 65% and 90% by weight, relative to the total weight of the composition.

According to one variant of the invention, the composition comprises a mixture of water and one or more cosmetically acceptable organic solvents chosen from C₁-C₄ alcohols, such as ethanol, isopropanol, tert-butanol or n-butanol; polyols such as glycerol, propylene glycol and polyethylene glycols, aromatic alcohols such as benzyl alcohol; and mixtures thereof.

According to this variant, the total content of organic solvent(s) is preferably between 0.1% and 40% by weight, more preferentially between 0.5% and 30% by weight, even more preferentially between 1 and 20% by weight, better still between 2% and 10% by weight, relative to the total weight of the composition.

The Propellants

The composition according to the present invention comprises one or more propellants.

The propellant(s) that may be used according to the invention are preferably chosen from liquefied gases such as dimethyl ether, chlorinated and/or fluorinated hydrocarbons such as trichlorofluoromethane, dichlorodifluoromethane, chlorodifluoromethane, 1,1,1,2-tetrafluoroethane, chloropentafluoroethane, 1-chloro-1,1-difluoroethane or 1,1-difluoroethane, or volatile hydrocarbons especially such as C₃-C₅ alkanes, for instance propane, isopropane, n-butane, isobutane or pentane; and mixtures thereof.

According to one preferred embodiment of the invention, the propellant(s) are chosen from volatile, optionally halogenated hydrocarbons, for example n-butane, propane, isobutane, pentane and halogenated derivatives thereof, dimethyl ether, and mixtures thereof; more preferentially from volatile hydrocarbons which are optionally halogenated; better still from C₃-C₅-alkanes, in particular propane, n-butane, isobutane, and mixtures thereof.

Preferably, the propellant(s) used are entirely dispersed in the composition before the first use of the composition. The expression “entirely dispersed in the composition” is intended to mean that the propellant(s) are entirely present in the droplets of oily phase and/or in the aqueous phase (for example dispersed in the aqueous phase in the form of micelles) of the composition in the form of a nanoemulsion—in other words the propellant(s) are emulsified in the composition according to the invention; or that the content of the propellant(s) not dispersed (i.e. not present within the nanoemulsion according to the invention) is less than 0.5% by weight relative to the total weight of the composition.

Preferably, the total content of the propellant(s) present in the composition is between 0.1% and 20% by weight, more preferentially between 0.5% and 15% by weight, even more preferentially between 1% and 7% by weight, better still less than or equal to 6% by weight, and even better still between 1% and 6% by weight, relative to the total weight of the composition.

Advantageously, the composition according to the invention is particularly clear when the total content of the propellant(s) present in the composition is less or equal to 6% by weight, more preferentially between 1% and 6% by weight, relative to the total weight of the composition.

The Fixing Polymers

Preferably, the composition according to the present invention also comprises one or more fixing polymers.

For the purposes of the invention, the term “fixing polymer” is intended to mean any polymer that is capable, by application to the hair, of giving a shape to the head of hair or of holding an already acquired shape.

All the anionic, amphoteric, cationic and non-ionic fixing polymers and mixtures thereof used in the art may be used in the compositions according to the present application.

Preferably, the fixing polymer(s) according to the invention are chosen from non-ionic fixing polymers and anionic fixing polymers, and mixtures thereof.

More preferentially, the fixing polymer(s) are chosen from non-ionic fixing polymers.

The anionic fixing polymers generally used are polymers containing groups derived from carboxylic, sulfonic or phosphoric acid, and have a number-average molecular weight of between approximately 500 and 5 000 000.

The carboxylic groups are provided by unsaturated mono- or dicarboxylic acid monomers, such as those corresponding to formula (III):

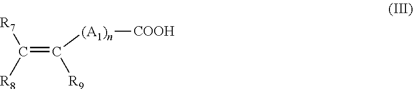

in which n is an integer from 0 to 10, A₁ denotes a methylene group, optionally connected to the carbon atom of the unsaturated group or to the adjacent methylene group when n is greater than 1, via a heteroatom, such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group or a —CH₂—COOH, phenyl or benzyl group.

In the abovementioned formula, a lower alkyl group preferably denotes a group having 1 to 4 carbon atoms and in particular methyl and ethyl groups.

The anionic fixing polymers containing carboxylic groups that are preferred according to the invention are:

A) copolymers of acrylic or methacrylic acid or salts thereof.

Mention may be made, among these polymers, of copolymers of acrylic or methacrylic acid with a monoethylenic monomer, such as ethylene, styrene, vinyl esters or acrylic or methacrylic acid esters, optionally grafted to a polyalkylene glycol, such as polyethylene glycol, and optionally crosslinked. Such polymers are described in particular in French patent No. 1 222 944 and German application No. 2330956, the copolymers of this type comprising an optionally N-alkylated and/or hydroxyalkylated acrylamide unit in their chain, as described in particular in the Luxembourgian patent applications Nos. 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM (INCI name: VP/acrylates/lauryl methacrylate copolymer), acrylic acid/ethyl acrylate/N-(t-butyl)acrylamide terpolymers, such as the products Ultrahold® Strong and Ultrahold® 8 sold by BASF (INCI name: Acrylates/t-butylacrylamide copolymer), methacrylic acid/ethyl acrylate/tert-butyl acrylate terpolymers, such as the products sold under the names Luvimer® 100 P or Luvimer® PRO 55 by BASF (INCI name: Acrylates copolymer), copolymers of methacrylic acid and of ethyl acrylate, such as the products sold under the names Luvimer® MAE or Luviflex® Soft by BASF (INCI name: Acrylates copolymer), acrylic acid/butyl acrylate/methyl methacrylate terpolymers, such as the product sold under the name Balance® CR by AkzoNobel (INCI name: Acrylates copolymer), or the copolymers of methacrylic acid and of methyl methacrylate sold under the name Eudragit® L 100 by Rohm Pharma (INCI name: Acrylates copolymer). Mention may also be made of branched block polymers containing (meth)acrylic acid monomers, such as the product sold under the name Fixate® G-100L by the company Lubrizol (INCI name AMP-acrylates/allyl methacrylate copolymer);

B) crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patents Nos. 1 222 944, 1 580 545, 2 265 782, 2 265 781, 1 564 110 and 2 439 798. Commercial products which fall into this category are the products Resyn® 28-2930 and 28-1310 sold by the company AkzoNobel (INCI names VA/crotonates/vinyl decanoate copolymer and VA/crotonates copolymer, respectively). Mention may also be made of the products Luviset® CA 66 sold by the company BASF, Aristoflex® A60 sold by the company Clariant (INCI name VA/crotonates copolymer) and Mexomere® PW or PAM sold by the company Chimex (INCI name VA/vinyl butyl benzoate/crotonates copolymer);

C) copolymers of C₄-C₈ monounsaturated carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, the anhydride functions of these copolymers optionally being monoesterified or monoamidated. Such polymers are described, in particular, in U.S. Pat. Nos. 2,047,398, 2,723,248 and 2,102,113, and patent GB 839 805. Commercial products are in particular those sold under the names Gantrez® AN or ES by the company ISP, such as Gantrez® ES 225 (INCI name Ethyl ester of PVM/MA copolymer) or Gantrez® ES 425L (INCI name Butyl ester of PVM/MA copolymer);

copolymers comprising (i) one or more maleic, citraconic or itaconic anhydride units and (ii) one or more monomers chosen from allyl or methallyl esters, optionally comprising one or more acrylamide, methacrylamide, a-olefin, acrylic or methacrylic ester, acrylic or methacrylic acid or vinylpyrrolidone groups in their chain,

the anhydride functions of these copolymers optionally being monoesterified or monoamidated.

These polymers are described, for example, in patents FR 2 350 384 and FR 2 357 241;

D) polyacrylamides comprising carboxylate groups.

The fixing polymers comprising units derived from sulfonic acid can be chosen from:

A′) homopolymers and copolymers comprising vinylsulfonic, styrenesulfonic, naphthalenesulfonic or acrylamidoalkylsulfonic units.

These polymers may be chosen especially from:

polyvinylsulfonic acid salts with a molecular weight of between 1,000 and 100 000 approximately, and also the copolymers with an unsaturated comonomer such as acrylic or methacrylic acids and esters thereof, and also acrylamide or derivatives thereof, vinyl ethers and vinylpyrrolidone;

polystyrenesulfonic acid salts such as the sodium salts that are sold for example under the name Flexan® II by AkzoNobel (INCI name Sodium polystyrene sulfonate). These compounds are described in patent FR 2198719;

polyacrylamidosulfonic acid salts, such as those mentioned in U.S. Pat. No. 4,128,631, and more particularly the polyacrylamidoethylpropanesulfonic acid sold under the name Rheocare® HSP-1180 by Cognis (INCI name polyacrylamidomethylpropane sulfonic acid);

B′) sulfonic polyesters, these polymers being advantageously obtained by polycondensation of at least one dicarboxylic acid, of at least one diol or of a mixture of diol and of diamine, and of at least one difunctional monomer comprising a sulfonic function. Among these polymers, mention may be made of:

linear sulfonic polyesters such as those described in patent applications U.S. Pat. Nos. 3,734,874, 3,779,993, 4,119,680, 4,300,580, 4,973,656, 5,660,816, 5,662,893 and 5,674,479. Such polymers are, for example, the products Eastman® AQ38S Polymer, Eastman® AQ55S Polymer and Eastman® AQ48 Ultra Polymer sold by the company Eastman Chemical (name Polyester-5) which are copolymers obtained from diethylene glycol, from 1,4-cyclohexanedimethanol, from isophthalic acid and from sulfoisophthalic acid salt;

branched sulfonic polyesters such as those described in patent applications WO 95/18191, WO 97/08261 and WO 97/20899. Such compounds are for example the products Eastman® AQ10D Polymer (name Polyester-13) or Eastman® AQ1350 Polymer provided by the company Eastman Chemical (name Polyester-13).

According to the invention, the anionic fixing polymers are preferably chosen from copolymers of acrylic acid, such as the acrylic acid/ethyl acrylate/N-tert-butylacrylamide terpolymers sold in particular under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold in particular under the name Resyn 28-2930 by the company AkzoNobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the names Gantrez® \ES 425L or ES 225 by the company ISP, the copolymers of methacrylic acid and of ethyl acrylate sold under the name Luvimer® MAE by the company BASF, and the vinyl acetate/crotonic acid copolymers sold under the name Luviset® CA 66 by the company BASF, and the vinyl acetate/crotonic acid copolymers grafted with polyethylene glycol sold under the name Aristoflex® A60 by the company Clariant, the vinylpyrrolidone/acrylic acid/lauryl methacrylate terpolymers sold under the name Acrylidone® LM by the company ISP, the polymer sold under the name Fixate® G-100L by the company Lubrizol, the vinyl acetate/crotonic acid/vinyl p-tert-butylbenzoate copolymers sold under the names Mexomere® PW or PAM by the company Chimex.

Preferably, when they are present, the composition according to the invention comprises the anionic fixing polymer(s) in an amount ranging from 0.1% to 20% by weight, preferentially from 0.2% to 15% by weight, and even more preferentially from 0.3% to 10% by weight, relative to the total weight of the composition.

The amphoteric fixing polymers which can be used in accordance with the invention can be chosen from polymers comprising B and C units randomly distributed in the polymer chain, where B denotes a unit deriving from a monomer comprising at least one basic nitrogen atom and C denotes a unit deriving from an acidic monomer comprising one or more carboxylic or sulfonic groups or else B and C can denote groups deriving from zwitterionic carboxybetaine or sulfobetaine monomers;

B and C may also denote a cationic polymer chain comprising primary, secondary, tertiary or quaternary amine groups, in which at least one of the amine groups bears a carboxylic or sulfonic group connected via a hydrocarbon-based group or alternatively B and C form part of a chain of a polymer comprising an α, β-dicarboxylic ethylene unit in which one of the carboxylic groups has been made to react with a polyamine comprising one or more primary or secondary amine groups.

The amphoteric fixing polymers corresponding to the definition given above which are more particularly preferred are chosen from the following polymers:

(1) copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537;

(2) polymers comprising units deriving:
a) from at least one monomer chosen from acrylamides and methacrylamides substituted on the nitrogen atom with an alkyl group,
b) from at least one acidic comonomer containing one or more reactive carboxylic groups, and
c) at least one basic comonomer such as esters with primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are more particularly chosen from acrylic acid, methacrylic acid, crotonic acid, itaconic acid, maleic acid and fumaric acid and alkyl monoesters, containing 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers of which the INCI name is Octylacrylamide/acrylates/butylaminoethyl methacrylate copolymer, such as the products sold under the names Amphomer®, Amphomer® LV71 or Balance® 47 by the company AkzoNobel, are particularly used;

(3) partially or completely acylated and crosslinked polyaminoamides deriving from polyaminoamides of general formula (IV):

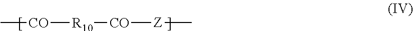

in which R₁₀ represents a divalent group derived from a saturated dicarboxylic acid, from an aliphatic mono- or dicarboxylic acid comprising an ethylenic double bond, from an ester of a lower alkanol having from 1 to 6 carbon atoms of these acids, or from a group deriving from the addition of any one of said acids with a bisprimary or bissecondary amine, and Z denotes a group deriving from a bisprimary, mono- or bissecondary polyalkylenepolyamine and preferably represents:

a) in proportions of from 60 mol % to 100 mol %, the group (V)

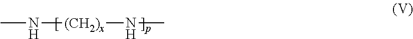

in which x=2 and p=2 or 3, or else x=3 and p=2,

this group deriving from diethylenetriamine, from triethylenetetramine or from dipropylenetriamine;

b) in proportions of from 0 to 40 mol %, the group (V) above in which x=2 and p=1 and which is derived from ethylenediamine, or the group deriving from piperazine:

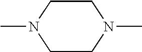

c) in the proportions of from 0 mol % to 20 mol %, the group —NH—(CH₂)₆—NH— deriving from hexamethylenediamine, these polyaminoamides being crosslinked by addition reaction of a bifunctional crosslinking agent chosen from epihalohydrins, diepoxides, dianhydrides or bisunsaturated derivatives, by means of from 0.025 to 0.35 mol of crosslinking agent per amine group of the polyaminoamide, and acylated by action of acrylic acid, chloroacetic acid or an alkane sultone or salts thereof.

The saturated carboxylic acids are preferably chosen from acids containing 6 to 10 carbon atoms, such as adipic acid, -2,2,4-trimethyladipic acid, 2,4,4-trimethyladipic acid and terephthalic acid, and acids bearing an ethylenic double bond, for instance acrylic, methacrylic and itaconic acids.

The alkane sultones used in the acylation are preferably propane sultone or butane sultone; the salts of the acylating agents are preferably the sodium or potassium salts.

(4) Polymers comprising zwitterionic units of formula (VI):

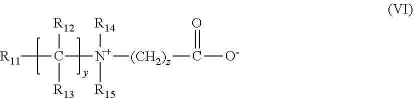

in which R₁₁ denotes a polymerizable unsaturated group, such as an acrylate, methacrylate, acrylamide or methacrylamide group, y and z represent an integer from 1 to 3, R₁₂ and R₁₃ represent a hydrogen atom or a methyl, ethyl or propyl group, and R₁₄ and R₁₅ represent a hydrogen atom or an alkyl group such that the sum of the carbon atoms in R₁₄ and R₁₅ does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate.

Mention may be made, by way of example, of methyl methacrylate/methyl dimethylcarboxymethylammonioethyl methacrylate copolymers, such as the product sold under the name Diaformer Z-301N or Z-301W by the company Clariant (INCI name Acrylates copolymer);

(5) polymers derived from chitosan comprising monomer units corresponding to the following formulae (D), (E) and (F):

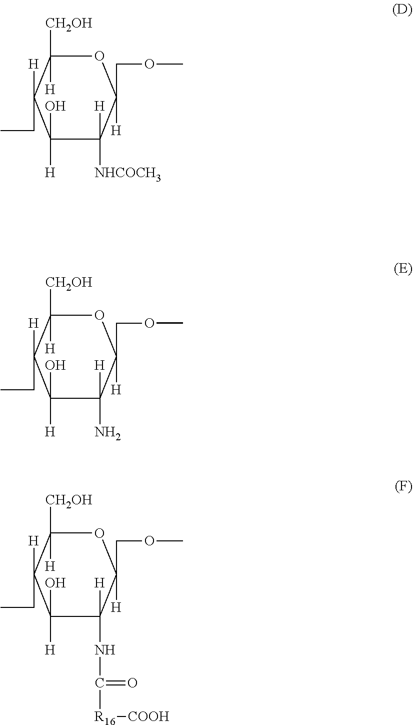

the unit (D) being present in proportions of between 0 and 30%, the unit (E) in proportions of between 5% and 50% and the unit (F) in proportions of between 30% and 90%, it being understood that, in this unit (F), R₁₆ represents a group of formula (VII):

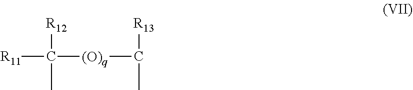

in which:

if q=0, R₁₁, R₁₂ and R₁₃, which may be identical or different, each represent a hydrogen atom, a methyl, hydroxyl, acetoxy or amino residue, a monoalkylamine residue or a dialkylamine residue that are optionally interrupted with one or more nitrogen atoms and/or optionally substituted with one or more amine, hydroxyl, carboxyl, alkylthio or sulfonic groups, an alkylthio residue in which the alkyl group bears an amino residue, at least one of the groups R₁₁, R₁₂ and R₁₃ being, in this case, a hydrogen atom;

or, if q=1, R₁₁, R₁₂ and R₁₃ each represent a hydrogen atom, and also the salts formed by these compounds with bases or acids;

(6) polymers containing units conforming to the general formula (VIII) are described, for example, in French patent FR 1 400 366:

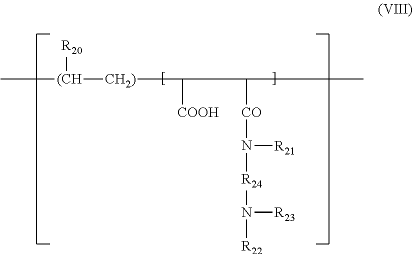

in which R₂₀ represents a hydrogen atom or a CH₃O—, CH₃CH₂O— or phenyl group, R₂₁ denotes a hydrogen atom or a lower alkyl group, such as methyl or ethyl, R₂₂ denotes a hydrogen atom or a lower C₁-C₆ alkyl group, such as methyl or ethyl, and R₂₃ denotes a lower C₁-C₆ alkyl group, such as methyl or ethyl, or a group corresponding to the formula: —R₂₄—N(R₂₂)₂, with R₂₄ representing a —CH₂—CH₂—, —CH₂—CH₂—CH₂, or —CH₂—CH(CH₃)— group and R₂₂ having the meanings given above;

(7) polymers derived from the N-carboxyalkylation of chitosan, such as N-(carboxymethyl)chitosan or N-(carboxybutyl)chitosan, such as, for example, the product sold under the name Chitoglycan by Sinerga SPA (INCI name: Carboxymethyl chitosan);

(8) amphoteric polymers of the -D-X-D-X type chosen from:
a) polymers obtained by the action of chloroacetic acid or sodium chloroacetate on compounds comprising at least one unit of formula (IX):

-D-X-D-X-D- (IX)

in which D denotes a group

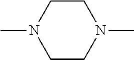

and X denotes the symbol E or E′, where E or E′, which may be identical or different, denote a divalent group that is an alkylene group with a straight or branched chain containing up to 7 carbon atoms in the main chain, which is unsubstituted or substituted with hydroxyl groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;

b) polymers of formula (X):

-D-X-D-X- (X)

in which D denotes a group

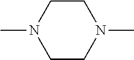

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers may also comprise other vinyl comonomers, such as vinylcaprolactam.

Mention will be made, among the abovementioned most particularly preferred amphoteric fixing polymers according to the invention, of those of family (3), such as the copolymers of which the INCI name is Octylacrylamide/acrylates/butylaminoethyl methacrylate copolymer, such as the products sold under the names Amphomer®, Amphomer® LV 71 or Balance® 47 by the company AkzoNobel, and those of family (4), such as methyl methacrylate/methyl dimethylcarboxymethylammonioethyl methacrylate copolymers, sold, for example, under the name Diaformer Z-301N or Z-301W by the company Clariant.

Preferably, when they are present, the composition according to the invention comprises the amphoteric fixing polymer(s) in an amount ranging from 0.1% to 20% by weight, preferentially from 0.2% to 15% by weight, and even more preferentially from 0.3% to 10% by weight, relative to the total weight of the composition.

The cationic fixing polymers that can be used according to the present invention are preferably chosen from polymers comprising primary, secondary, tertiary and/or quaternary amine groups which are part of the polymer chain or directly connected thereto, and which have a molecular weight of between 500 and approximately 5 000 000 and preferably between 1000 and 3 000 000.

Among these polymers, mention may more in particular be made of the following cationic polymers:

(1) homopolymers or copolymers derived from acrylic or methacrylic esters or amides and comprising at least one of the units having the following formulae:

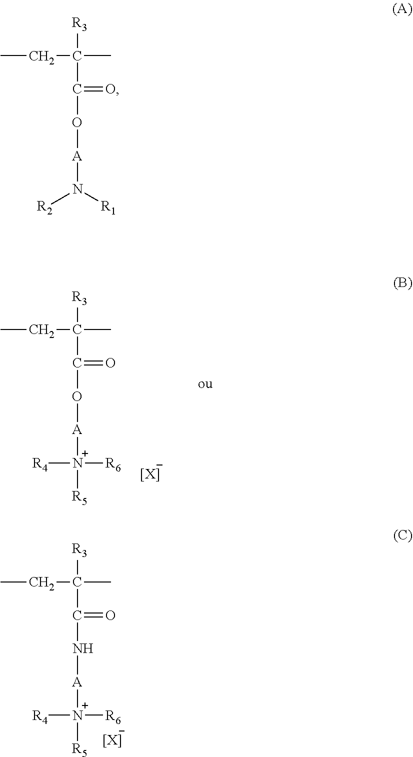

wherein:

R₃ denotes a hydrogen atom or a CH₃ group;

A is a linear or branched alkyl group comprising from 1 to 6 carbon atoms or a hydroxyalkyl group comprising from 1 to 4 carbon atoms;

R₄, R₅, R₆, which may be identical or different, represent an alkyl group having from 1 to 18 carbon atoms or a benzyl group;

R₁ and R₂, which may be identical or different, each represent a hydrogen atom or an alkyl group having from 1 to 6 carbon atoms;

X denotes a methosulfate anion or a halide such as chloride or bromide.
The copolymers of family (1) also contain one or more units which derive from comonomers that can be chosen from the family of acrylamides, methacrylamides, diacetone-acrylamides, acrylamides and methacrylamides substituted on the nitrogen with C₁-C₄ alkyl groups, groups derived from acrylic or methacrylic acids or esters thereof, vinyllactams such as vinylpyrrolidone or vinylcaprolactam, or vinyl esters.

Thus, among these comonomers of family (1), mention may be made of:

copolymers of acrylamide and of dimethylaminoethyl methacrylate quaternized with dimethyl sulfate with a dimethyl halide, such as that sold under the name Hercofloc® by the company Hercules,

copolymers of acrylamide and of methacryloyloxyethyltrimethylammonium chloride described for example in Patent Application EP-A-080976 and sold under the name Bina Quat P 100 by the company Ciba Geigy,

the copolymer of acrylamide and of methacryloyloxyethyltrimethylammonium methosulfate such as that sold under the name Reten by the company Hercules,

vinylpyrrolidone/dialkylaminoalkyl acrylate or methacrylate copolymers which are optionally quaternized, such as the products sold under the name Gafquat® by the company ISP, such as for example Gafquat® 734 or Gafquat® 755 or else the products called Copolymer® 845, 958 and 937. These polymers are described in detail in French Patents 2 077 143 and 2 393 573,

polymers comprising a fatty chain and a vinylpyrrolidone unit, such as the products sold under the name Stylèze W20 and Stylèze W10 by the company ISP,

dimethylaminoethyl methacrylate/vinylcaprolactam/vinyl-pyrrolidone terpolymers such as the product sold under the name Gaffix VC 713 by the company ISP, and

quaternized vinylpyrrolidone/dimethylaminopropyl methacrylamide copolymers such as the products sold under the name Gafquat® HS 100 by the company ISP;

(2) cationic guar gums, preferably comprising a quaternary ammonium, such as those described in U.S. Pat. Nos. 3,589,578 and 4,031,307, such as guar gums containing trialkylammonium cationic groups. Such products are sold in particular under the tradenames Jaguar C13 S, Jaguar C 15, Jaguar C 17 by the company Meyhall;

(3) quaternary copolymers of vinylpyrrolidone and of vinylimidazole;

(4) chitosans or salts thereof; the salts that can be used are in particular the acetate, lactate, glutamate, gluconate or pyrrolidonecarboxylate of chitosan.

Among these compounds, mention may be made of chitosan having a degree of deacetylation of 90.5% by weight, sold under the name Kytan Brut Standard by the company Aber Technologies, the chitosan pyrrolidonecarboxylate sold under the name Kytamer® PC by the company Amerchol;

(5) cationic cellulose derivatives such as copolymers of cellulose or of cellulose derivatives grafted with a water-soluble monomer comprising a quaternary ammonium, and described in particular in U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium salt, methacrylamidopropyltrimethylammonium salt or dimethyldiallylammonium salt.

The commercially available products corresponding to this definition are more particularly the products sold under the name Celquat L 200 and Celquat H 100 by the company National Starch.

The non-ionic fixing polymers that may be used according to the present invention are chosen, for example, from:

polyalkyloxazolines;

vinyl acetate homopolymers,

vinyl acetate copolymers, such as, for example, copolymers of vinyl acetate and of acrylic ester, copolymers of vinyl acetate and of ethylene, or copolymers of vinyl acetate and of maleic ester, for example of dibutyl maleate;

acrylic ester homopolymers and copolymers, such as, for example, copolymers of alkyl acrylates and of alkyl methacrylates, such as the products provided by Rohm GmbH under the name Eudragit® NE 30 D (INCI name: Acrylates copolymer);

copolymers of acrylonitrile and of a non-ionic monomer chosen, for example, from butadiene and alkyl (meth)acrylates;

styrene homopolymers;

styrene copolymers, such as, for example, copolymers of styrene, of alkyl acrylate and of alkyl methacrylate; copolymers of styrene and of butadiene; or copolymers of styrene, of butadiene and of vinylpyridine;

polyamides;

vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF (INCI name PVP);

vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/vinylimidazole terpolymers, for instance the product sold under the name Luviset® Clear by the company BASF (INCI name VP/methacrylamide/vinyl imidazole copolymer).

The alkyl groups of the non-ionic polymers mentioned above preferably contain from 1 to 6 carbon atoms.

Preferably, when they are present, the composition according to the invention comprises the non-ionic fixing polymer(s) in an amount ranging from 0.1% to 20% by weight, preferentially from 0.2% to 15% by weight, and even more preferentially from 0.3% to 10% by weight, relative to the total weight of the composition.

Use may also be made, according to the invention, of fixing polymers of grafted silicone type comprising a polysiloxane portion and a portion composed of a non-silicone organic chain, one of the two portions constituting the main chain of the polymer and the other being grafted to said main chain.

These polymers are described, for example, in the patent applications EP-A-0 412 704, EP-A-0 412 707, EP-A-0 640 105 and WO 95/00578, EP-A-0 582 152 and WO 93/23009, and the U.S. Pat. Nos. 4,693,935, 4,728,571 and 4,972,037.

These polymers can be amphoteric, cationic, anionic or non-ionic and they are preferably anionic or non-ionic.

Such polymers are, for example, copolymers that may be obtained by free radical polymerization from the monomer mixture formed from:

a) 50% to 90% by weight of tert-butyl acrylate,

b) 0% to 40% by weight of acrylic acid,

c) 5% to 40% by weight of a silicone macromer of formula:

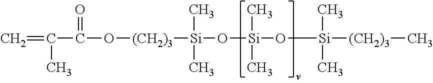

where v is a number ranging from 5 to 700, the percentages by weight being calculated relative to the total weight of the monomers.

Other examples of grafted silicone polymers are in particular polydimethylsiloxanes (PDMSs) to which mixed polymer units of the poly((meth)acrylic acid) type and of the poly(alkyl (meth)acrylate) type are grafted via a thiopropylene-type connecting link and polydimethylsiloxanes (PDMSs) to which polymer units of the poly(isobutyl (meth)acrylate) type are grafted via a thiopropylene-type connecting link.

Grafted silicone polymers are, for example, sold under the names Silicone Plus Polymer® VS80 and VA70 by 3M (INCI names Polysilicone-8 and Polysilicone-7 respectively).

Another type of silicone fixing polymer that may be mentioned is the product Luviflex® Silk sold by BASF (INCI name PEG/PPG-25/25 dimethicone/acrylates copolymer).

Preferably, when they are present, the composition according to the invention comprises the fixing polymer(s) of grafted silicone type in an amount ranging from 0.1% to 20% by weight, preferentially from 0.2% to 15% by weight, and even more preferentially from 0.3% to 10% by weight, relative to the total weight of the composition.

Functionalized or non-functionalized, silicone or non-silicone, non-ionic, anionic, cationic or amphoteric polyurethanes or mixtures thereof may also be used as fixing polymers.

The polyurethanes particularly targeted by the present invention are those described in the patent applications EP 0 751 162, EP 0 637 600, EP 0 648 485 and FR 2 743 297, of which the applicant company is the proprietor, and also in the patent applications EP 0 656 021 and WO 94/03510 of BASF and EP 0 619 111 of National Starch.

Mention may be made, as polyurethanes particularly suitable in the present invention, of the products sold under the names Luviset PUR® and Luviset® Si PUR by the company BASF (INCI names Polyurethane-1 and Polyurethane-6 respectively).

Preferably, when they are present, the composition according to the invention comprises the polyurethane(s) in an amount ranging from 0.1% to 20% by weight, preferentially from 0.2% to 15% by weight, and even more preferentially from 0.3% to 10% by weight, relative to the total weight of the composition.

Preferably, the composition according to the invention also comprises one or more fixing polymers chosen from non-ionic fixing polymers, anionic fixing polymers, and mixtures thereof; preferentially chosen from non-ionic fixing polymers; more preferentially chosen from vinyllactam homopolymers such as homopolymers of vinylpyrrolidone and polyvinylcaprolactam, and vinyllactam copolymers such as poly(vinylpyrrolidone/vinyllactam) copolymers and poly(vinylpyrrolidone/vinyl acetate) copolymers; and mixtures thereof.

Preferably, the composition according to the invention is free of cationic polymer.

Preferably, when they are present, the composition according to the invention comprises the fixing polymer(s) in a total amount ranging from 0.1% to 20% by weight, preferentially from 0.2% to 15% by weight, and even more preferentially from 0.5% to 10% by weight, better still from 1% to 5% by weight, relative to the total weight of the composition.

The Additional Surfactants

Preferably, the composition according to the present invention also comprises one or more additional surfactants chosen from anionic surfactants, amphoteric or zwitterionic surfactants, cationic surfactants, non-ionic surfactants other than the non-ionic surfactants of formula (I) as previously described, and mixtures thereof.

The anionic surfactants may be chosen especially from anionic derivatives of proteins of plant origin or of silk proteins, phosphates and alkyl phosphates, carboxylic acids and carboxylates, sulfosuccinates, amino acid derivatives, alkyl sulfates, alkyl ether sulfates, sulfonates, isethionates, taurates, alkyl sulfoacetates, polypeptides, anionic derivatives of alkyl polyglucoside, soaps (fatty acid salts), and mixtures thereof.

a) Anionic derivatives of proteins of plant origin are protein hydrolysates bearing a hydrophobic group, it being possible for said hydrophobic group to be naturally present in the protein or to be added by reaction of the protein and/or of the protein hydrolysate with a hydrophobic compound. The proteins are of plant origin or are derived from silk, and the hydrophobic group may in particular be a fatty chain, for example an alkyl chain comprising from 10 to 22 carbon atoms. As anionic derivatives of proteins of plant origin, mention may more particularly be made of apple, wheat, soybean or oat protein hydrolysates comprising an alkyl chain containing from 10 to 22 carbon atoms, and salts thereof. The alkyl chain may especially be a lauryl chain and the salt may be a sodium, potassium and/or ammonium salt.

Thus, as protein hydrolysates bearing a hydrophobic group, mention may be made, for example, of salts of protein hydrolysates where the protein is a silk protein modified with lauric acid, such as the product sold under the name Kawa Silk by Kawaken; salts of protein hydrolysates where the protein is a wheat protein modified with lauric acid, such as the potassium salt sold under the name Aminofoam W OR by Croda (CTFA name: potassium lauroyl wheat amino acids) and the sodium salt sold under the name Proteol LW 30 by the company SEPPIC (CTFA name: sodium lauroyl wheat amino acids); salts of protein hydrolysates where the protein is an oat protein comprising an alkyl chain containing from 10 to 22 carbon atoms and more especially salts of protein hydrolysates where the protein is an oat protein modified with lauric acid, such as the sodium salt sold under the name Proteol OAT (30% aqueous solution) by the company SEPPIC (CTFA name: sodium lauroyl oat amino acids); or salts of apple protein hydrolysates comprising an alkyl chain containing from 10 to 22 carbon atoms, such as the sodium salt sold under the name Proteol APL (30% aqueous glycol solution) by the company SEPPIC (CTFA name: Sodium Cocoyl Apple amino acids). Mention may also be made of the mixture of lauroyl amino acids (aspartic, glutamic, glycine, alanine) neutralized with sodium N-methylglycinate sold under the name Proteol SAV 50 S by the company SEPPIC (CTFA name: Sodium Cocoyl amino acids).

b) Examples of phosphates and alkyl phosphates that may be mentioned include monoalkyl phosphates and dialkyl phosphates, such as the lauryl monophosphate sold under the name MAP 20® by the company Kao Chemicals, the potassium salt of dodecylphosphoric acid, a mixture of monoester and diester (predominantly diester) sold under the name Crafol AP-31® by the company Cognis, the mixture of octylphosphoric acid monoester and diester sold under the name Crafol AP-20® by the company Cognis, the mixture of ethoxylated (7 mol of EO) phosphoric acid monoester and diester of 2-butyloctanol, sold under the name Isofol 12 7 EO-Phosphate Ester® by the company Condea, the potassium or triethanolamine salt of mono(C12-C13)alkyl phosphate sold under the references Arlatone MAP 230K-40® and Arlatone MAP 230T-60® by the company Uniqema, the potassium lauryl phosphate sold under the name Dermalcare MAP XC-99/09® by the company Rhodia Chimie, and the potassium cetyl phosphate sold under the name Arlatone MAP 160K by the company Uniqema.

c) As carboxylic acids and carboxylates, mention may for example be made of amide ether carboxylates (AECs), such as sodium laurylamide ether carboxylate (3 EO), sold under the name Akpyo Foam 30® by the company Kao Chemicals, polyoxyethylenated carboxylic acid salts, such as oxyethylenated (6 EO) sodium lauryl ether carboxylate (C12-14-16 65/25/10) sold under the name Akpyo Soft 45 NV® by the company Kao Chemicals, polyoxyethylenated and carboxymethylated fatty acids of olive oil origin, sold under the name Olivem 400® by the company Biologia e Tecnologia, oxyethylenated (6 EO) sodium tridecyl ether carboxylate sold under the name Nikkol ECTD-6NEX® by the company Nikkol, fatty acids having a C₆ to C₂₂ alkyl chain, such as stearic acid, and fatty acid salts (soaps) having a C₆ to C₂₂ alkyl chain, neutralized with an organic or mineral base such as potassium hydroxide, sodium hydroxide, triethanolamine, N-methyl glucamine, lysine and arginine.

d) Amino acid derivatives that may especially be mentioned include alkaline salts of amino acids, such as:

sarcosinates, for instance sodium cocoyl sarcosinate, the sodium lauroyl sarcosinate sold under the name Sarkosyl NL 97® by the company Ciba or sold under the name Oramix L 30® by the company SEPPIC, sodium myristoyl sarcosinate sold under the name Nikkol Sarcosinate MN® by the company Nikkol, and sodium palmitoyl sarcosinate sold under the name Nikkol Sarcosinate PN® by the company Nikkol;

alaninates, for instance sodium N-lauroyl N-methyl amidopropionate sold under the name Sodium Nikkol Alaninate LN 30® by the company Nikkol, or sold under the name Alanone ALE® by the company Kawaken, and triethanolamine N-lauroyl N-methyl alanine sold under the name Alanone Alta® by the company Kawaken;

glutamates, for instance triethanolamine monococoyl glutamate sold under the name Acylglutamate CT-12® by the company Ajinomoto, triethanolamine lauroyl glutamate sold under the name Acylglutamate LT-12® by the company Ajinomoto, aspartates, for instance the mixture of triethanolamine N-lauroyl aspartate/triethanolamine N-myristoyl aspartate sold under the name Asparack® by the company Mitsubishi;

glycine derivatives (glycinates), for instance the sodium N-cocoyl glycinate sold under the names Amilite GCS-12® and Amilite GCK 12 by the company Ajinomoto;

citrates, such as the oxyethylenated (9 mol) citric monoester of cocoyl alcohols sold under the name Witconol EC 1129 by the company Goldschmidt, and galacturonates such as sodium dodecyl D-galactoside uronate sold by the company Soliance.

e) Examples of sulfosuccinates that may be mentioned include the oxyethylenated (3 EO) lauryl alcohol monosulfosuccinate (70/30 C12/C14) sold under the names Setacin 103 Special® and Rewopol SB-FA 30 K 4® by the company Witco, the disodium salt of a hemisulfosuccinate of C12-C14 alcohols, sold under the name Setacin F Special Paste® by the company Zschimmer Schwarz, the oxyethylenated (2 EO) disodium oleamidosulfosuccinate sold under the name Standapol SH 135® by the company Cognis, the oxyethylenated (5 EO) laurylamide monosulfosuccinate sold under the name Lebon A-5000® by the company Sanyo, the oxyethylenated (10 EO) disodium salt of lauryl citrate monosulfosuccinate sold under the name Rewopol SB CS 50® by the company Witco, and the ricinoleic monoethanolamide monosulfosuccinate sold under the name Rewoderm S 1333® by the company Witco. Polydimethylsiloxane sulfosuccinates may also be used, such as the disodium PEG-12 dimethicone sulfosuccinate sold under the name Mackanate-DC30 by the company Maclntyre.

f) Examples of alkyl sulfates that may be mentioned include triethanolamine lauryl sulfate (CTFA name: TEA lauryl sulfate), such as the product sold by Huntsman under the name Empicol TL40 FL or the product sold by Cognis under the name Texapon T42, which products are at 40% in aqueous solution. Mention may also be made of ammonium lauryl sulfate (CTFA name: ammonium lauryl sulfate), such as the product sold by Huntsman under the name Empicol AL 30FL, which is at 30% in aqueous solution.

g) Examples of alkyl ether sulfates that may be mentioned include sodium lauryl ether sulfate (CTFA name: sodium laureth sulfate), such as the product sold under the names Texapon N40 and Texapon AOS 225 UP by the company Cognis, or ammonium lauryl ether sulfate (CTFA name: ammonium laureth sulfate), such as the product sold under the name Standapol EA-2 by the company Cognis.

h) Examples of sulfonates that may be mentioned include α-olefinsulfonates, such as the sodium α-olefinsulfonate (C14-C16), sold under the name Bio-Terge AS-40® by the company Stepan, sold under the names Witconate AOS Protégé® and Sulframine AOS PH 12® by the company Witco or sold under the name Bio-Terge AS-40 CG® by the company Stepan, secondary sodium olefinsulfonate, sold under the name Hostapur SAS 30® by the company Clariant; or linear alkylarylsulfonates, such as sodium xylenesulfonate, sold under the names Manrosol SXS30®, Manrosol SXS40® and Manrosol SXS93® by the company Manro.

i) Isethionates that may be mentioned include (C₈-C₁₈)acylisethionates, for instance sodium cocoyl isethionate, such as the product sold under the name Jordapon CI P® by the company Jordan.

j) Taurates that may be mentioned include the salts (in particular sodium salt) of palm kernel oil methyltaurate sold under the name Hostapon CT Paste® by the company Clariant; N-(C8-C18)acyl N-methyltaurates, for instance the sodium N-cocoyl N-methyltaurate sold under the name Hostapon LT-SF® by the company Clariant or sold under the name Nikkol CMT-30-T® by the company Nikkol, and the sodium palmitoyl methyltaurate sold under the name Nikkol PMT® by the company Nikkol.

k) The anionic derivatives of (C₈-C₁₈)alkyl polyglucosides may especially be citrates, tartrates, sulfosuccinates, carbonates and ethers of glycerol obtained from alkyl polyglucosides. Mention may be made, for example, of the sodium salt of cocoyl polyglucoside (1,4) tartaric ester, sold under the name Eucarol AGE-ET® by the company Cesalpinia, the disodium salt of cocoyl polyglucoside (1,4) sulfosuccinic ester, sold under the name Essai 512 MP® by the company SEPPIC, or the sodium salt of cocoyl polyglucoside (1,4) citric ester, sold under the name Eucarol AGE-EC® by the company Cesalpinia.

1) The soaps are obtained from a fatty acid which is partially or completely saponified (neutralized) with a basic agent. These are alkali metal or alkaline-earth metal soaps or soaps of organic bases. Use may be made, as fatty acids, of saturated, linear or branched fatty acids comprising from 8 to 30 carbon atoms and preferably comprising from 8 to 22 carbon atoms. This fatty acid may be chosen in particular from palmitic acid, stearic acid, myristic acid and lauric acid, and mixtures thereof.

Examples of basic agents that may be used include alkali metal hydroxides (sodium hydroxide and potassium hydroxide), alkaline-earth metal hydroxides (for example magnesium hydroxide), ammonium hydroxide or else organic bases, such as triethanolamine, N-methylglucamine, lysine and arginine.

The soaps may especially be fatty acid alkali metal salts, the basic agent being an alkali metal hydroxide and preferably potassium hydroxide (KOH).

The amount of basic agent must be sufficient for the fatty acid to be at least partially neutralized.

More preferentially, the anionic surfactant(s) are chosen from alkyl sulfates, alkyl ether sulfates such as sodium lauryl ether sulfate, phosphates, alkylphosphates such as potassium cetylphosphate, amino acid derivatives, in particular sarcosine derivatives (sarcosinates), such as sodium cocoyl sarcosinate, soaps such as sodium stearate, carboxylic acids such as stearic acid, and mixtures thereof.

Even more preferentially, the anionic surfactant(s) are chosen from phosphates, alkylphosphates such as potassium cetylphosphate, sarcosine derivatives (sarcosinates), such as sodium cocoyl sarcosinate, soaps such as sodium stearate, carboxylic acids such as stearic acid, and mixtures thereof.

The term “cationic surfactant” is intended to mean a surfactant that is positively charged when it is contained in the composition according to the invention. This surfactant may bear one or more positive permanent charges or may contain one or more cationizable functions in the composition according to the invention.

The cationic surfactants may be chosen in particular from primary, secondary or tertiary fatty amines, which are optionally polyalkylenated, or salts thereof, and quaternary ammonium salts, and mixtures thereof.

The fatty amines comprise in general at least one C₈-C₃₀ hydrocarbon-based chain.

Examples of quaternary ammonium salts that may especially be mentioned include:

those corresponding to general formula (XI) below:

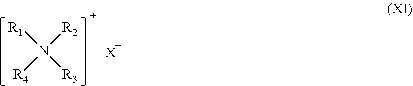

in which the groups of R₁ to R₄, which may be identical or different, represent a linear or branched aliphatic group comprising from 1 to 30 carbon atoms, or an aromatic group such as aryl or alkylaryl, at least one of the groups R₁ to R₄ denoting a linear or branched aliphatic radical comprising from 8 to 30 carbon atoms, preferably from 12 to 24 carbon atoms. The aliphatic groups may comprise heteroatoms such as, in particular, oxygen, nitrogen, sulfur and halogens. The aliphatic groups are for example chosen from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆) alkyl, (C₁₂-C₂₂) alkyl acetate and C₁-C₃₀ hydroxyalkyl groups; X⁻ is an anion chosen from the group of the halides, phosphates, acetates, lactates, (C₁-C₄)alkyl sulfates, and (C₁-C₄) alkyl- or (C₁-C₄)alkylarylsulfonates.

Among the quaternary ammonium salts of formula (XI), the ones that are preferred are, on the one hand, tetraalkylammonium salts, for instance the dialkyldimethylammonium or alkyltrimethylammonium salts in which the alkyl group comprises approximately from 12 to 22 carbon atoms, in particular behenyltrimethylammonium, distearyldimethylammonium, cetyltrimethylammonium or benzyldimethylstearylammonium salts, or else, on the other hand, the palmitylamidopropyltrimethylammonium salts, the stearamidopropyltrimethylammonium salts, the stearamidopropyldimethylcetearylammonium salts, or the stearamidopropyldimethyl(myristyl acetate)ammonium salts sold under the name Ceraphyl® 70 from the company Van Dyk. It is preferred in particular to use the chloride salts of these compounds;

quaternary ammonium salts of imidazoline, for instance those of formula (XII) below:

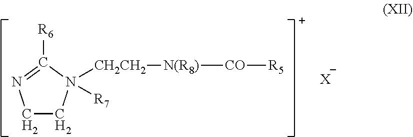

in which Rs represents an alkenyl or alkyl group comprising from 8 to 30 carbon atoms, for example derived from tallow fatty acids, R₆ represents a hydrogen atom, a C₁-C₄ alkyl group or an alkenyl or alkyl group comprising from 8 to 30 carbon atoms, R₇ represents a C₁-C₄ alkyl group, R₈ represents a hydrogen atom or a C₁-C₄ alkyl group and X⁻ is an anion chosen from the group of the halides, phosphates, acetates, lactates, alkyl sulfates, alkylsulfonates or alkylarylsulfonates, the alkyl and aryl groups of which preferably comprise, respectively, from 1 to 20 carbon atoms and from 6 to 30 carbon atoms. Preferably, R₅ and R₆ denote a mixture of alkenyl or alkyl groups comprising from 12 to 21 carbon atoms, for example derived from tallow fatty acids, R₇ denotes a methyl group and R₈ denotes a hydrogen atom. Such a product is sold, for example, under the name Rewoquat® W 75 by the company Rewo;

—di—or triquaternary ammonium salts, in particular of formula (XIII):

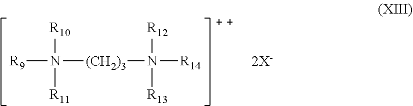

in which R₉ denotes an alkyl radical comprising approximately from 16 to 30 carbon atoms, which is optionally hydroxylated and/or optionally interrupted with one or more oxygen atoms, Rio is chosen from hydrogen or an alkyl radical comprising from 1 to 4 carbon atoms or a group (R₉ₐ)(R₁₀ₐ)(R₁₁ₐ)N—(CH₂)₃,

R₉ₐ, R₁₀ₐ, R₁₁ₐ, R₁₁, R₁₂, R₁₃ and R₁₄, which may be identical or different, are chosen from hydrogen or an alkyl radical comprising from 1 to 4 carbon atoms, and X⁻ is an anion chosen from the group of halides, acetates, phosphates, nitrates, (C₁-C₄)alkyl sulfates, (C₁-C₄)alkyl sulfonates and (C₁-C₄)alkylaryl sulfonates, and in particular methyl sulfate and ethyl sulfate. Such compounds are, for example, Finquat CT-P, sold by the company Finetex (Quaternium 89), and Finquat CT, sold by the company Finetex (Quaternium 75);

quaternary ammonium salts containing at least one ester function, such as those of formula (XIV) below:

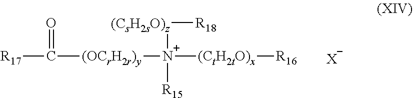

in which:

R₁₅ is chosen from C₁-C₆ alkyl groups and C₁-C₆ hydroxyalkyl or dihydroxyalkyl groups;

R₁₆ is chosen from:

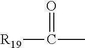

the group

groups R₂₀, which are linear or branched, saturated or unsaturated C₁-C₂₂ hydrocarbon-based groups,

a hydrogen atom;

R₁₈ is chosen from:

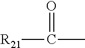

the group

groups R₂₂, which are linear or branched, saturated or unsaturated C₁-C₆ hydrocarbon-based groups,

a hydrogen atom;

R₁₇, R₁₉ and R₂₁, which may be identical or different, are chosen from linear or branched, saturated or unsaturated C₇-C₂₁ hydrocarbon-based groups;

r, s and t, which may be identical or different, are integers ranging from 2

to 6;

y is an integer ranging from 1 to 10;

x and z, which may be identical or different, are integers ranging from 0

to 10;

X⁻ is a simple or complex, organic or mineral anion; with the proviso that the sum x+y+z is from 1 to 15, that when x is 0 then R₁₆ denotes R₂₀, and that when z is 0 then R₁₈ denotes R₂₂.

The alkyl groups R₁₅ may be linear or branched, and more particularly linear.

Preferably, R₁₅ denotes a methyl, ethyl, hydroxyethyl or dihydroxypropyl group, and more particularly a methyl or ethyl group.

Advantageously, the sum x+y+z is from 1 to 10.

When R₁₆ is a hydrocarbon-based group R2o, it may be long and contain from 12 to 22 carbon atoms, or may be short and contain from 1 to 3 carbon atoms.

When R₁₈ is a hydrocarbon-based group R₂₂, it preferably contains 1 to 3 carbon atoms.

Advantageously, R₁₇, R₁₉ and R_{21,} which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride or methyl sulfate.

Use may be made more particularly in the composition according to the invention of the ammonium salts of formula (XIV) in which:

R₁₅ denotes a methyl or ethyl group;

x and y are equal to 1;

z is equal to 0 or 1;

r, s and t are equal to 2;

R₁₆ is chosen from:

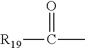

the group

methyl, ethyl or C₁₄-C₂₂ hydrocarbon-based groups,

a hydrogen atom;

R₁₈ is chosen from:

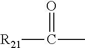

the group

a hydrogen atom;

R₁₇, R₁₉ and R₂₁, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Examples that may be mentioned include the compounds of formula (IV) such as the diacyloxyethyldimethylammonium, diacyloxy-ethylhydroxyethylmethylammonium, monoacyloxyethyldihydroxyethylmethylammonium, triacyloxyethyl-methylammonium and monoacyloxyethylhydroxyethyldimethyl-ammonium salts (chloride or methyl sulfate in particular), and mixtures thereof. The acyl groups preferably contain from 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may be made of behenoylhydroxypropyltrimethylammonium chloride sold by KAO under the name Quatarmin BTC 131.

Preferably, the ammonium salts containing at least one ester function contain two ester functions.

Among the quaternary ammonium salts containing at least one ester function, which may be used, it is preferred to use dipalmitoyl-ethylhydroxyethylmethylammonium salts.

The cationic surfactant(s) are preferably chosen from those of formula (XI) or those of formula (XIV), and even more preferentially from those of formula (XI).

Most particularly preferably, the cationic surfactant(s) that may be used according to the invention are chosen from those of formula (XI), more preferentially from behenyltrimethylammonium salts, cetyltrimethylammonium salts, and a mixture of these compounds, and even more preferentially from behenyltrimethylammonium chloride, cetyltrimethylammonium chloride, and a mixture of these compounds.

Preferentially, the cationic surfactant(s) that may be used in the composition according to the invention are chosen from the cationic surfactants of formula (XI).

The amphoteric or zwitterionic surfactants may be chosen in particular from derivatives of secondary or tertiary aliphatic amines, which are optionally quaternized, in which the aliphatic group is a linear or branched chain comprising from 8 to 22 carbon atoms, said amine derivatives containing at least one anionic group such as, for example, a carboxylate, sulfonate, sulfate, phosphate or phosphonate group. Mention may in particular be made of (C₈-C₂₀)alkylbetaines, sulfobetaines, (C₈-C₂₀ alkyl)amido (C₃-C₈ alkyl)betaines or (C₈-C₂₀ alkyl)amido(C₆-C₈ alkyl)sulfobetaines.

Among the derivatives of secondary or tertiary aliphatic amines, which are optionally quaternized, as defined above, that may be used, mention may also be made of the compounds having the respective structures (B1) and (B2) below:

Ra—C(O)—NHCH₂CH₂N⁺(Rb)(Rc)—CH₂C(O)O⁻, M⁺, X⁻ (B1)

In which formula (B1):

Ra represents a C₁₀-C₃₀ alkyl or alkenyl group derived from an acid RaCOOH, preferably present in hydrolyzed coconut oil, or a heptyl, nonyl or undecyl group;

Rb represents a beta-hydroxyethyl group; and

Rc represents a carboxymethyl group;

M⁺ represents a cationic counterion derived from an alkali metal or alkaline-earth metal, such as sodium, an ammonium ion or an ion derived from an organic amine, and

X⁻ represents an organic or inorganic anionic counterion, preferably chosen from halides, acetates, phosphates, nitrates, (C₁-C₄)alkyl sulfates, (C₁-C₄)alkyl- or (C₁-C₄)alkylaryl sulfonates, in particular methyl sulfate and ethyl sulfate;

or else M⁺ and X⁻ are absent;

Ra′—C(O)—NH—CH₂—CH₂—N(B)(B′) (B2)

in which formula:

B represents the group —CH₂—CH₂—O—X′;

B′ represents the group —(CH₂)_{z}Y′, with z=1 or 2;

X′ represents the group —CH₂—C(O)OH, —CH₂—C(O)OZ′, —CH₂—CH₂—C(O)OH, —CH₂—CH₂—C(O)OZ′, or a hydrogen atom;

Y′ represents the group —C(O)OH, —C(O)OZ′, —CH₂—CH(OH)—SO₃H or the group —CH₂—CH(OH)—SO₃—Z′;

Z′ represents a cationic counterion derived from an alkali metal or an alkaline-earth metal, such as sodium, an ammonium ion or an ion derived from an organic amine;

Ra′ represents a C₁₀-C₃₀ alkyl or C₁₀-C₃₀ alkenyl group of an acid Ra′—COOH preferably present in hydrolyzed linseed oil or coconut oil, an alkyl groups, in particular a C₁₇ alkyl group and the iso form thereof, an unsaturated C₁₇ group.

The compounds of this type are classified in the CTFA dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the tradename Miranol® C2M Concentrate.

Use may also be made of compounds of formula (B′2):

Ra″—NH—CH(Y″)—(CH₂)ₙ—C(O)NH(CH₂)_{2′}—N(Rd)(Re) (B′2)

in which formula:

Y″ represents the group —C(O)OH, —C(O)OZ″, —CH₂—CH(OH)—SO₃H or the group —CH₂—CH(OH)—SO₃—Z″;

Rd and Re, independently of one another, represent a C₁-C₄ alkyl or hydroxyalkyl radical;

Z″ represents a cationic counterion derived from an alkali metal or an alkaline-earth metal, such as sodium, an ammonium ion or an ion derived from an organic amine;

Ra″ represents a C₁₀-C₃₀ alkyl or alkenyl group of an acid Ra″—C(O)OH preferably present in hydrolysed linseed oil or coconut oil;

n and n′, independently of one another, denote an integer ranging from 1 to 3.

Among the compounds of formula (B′2), mention may be made of the compound classified in the CTFA dictionary under the name sodium diethylaminopropyl cocoaspartamide and sold by the company Chimex under the name Chimexane HB.

Among the amphoteric or zwitterionic surfactants mentioned above, use is preferably made of (C₈-C₂₀ alkyl)betaines such as cocoylbetaine, (C₈-C₂₀ alkyl)amido(C₂-C₈ alkyl)betaines such as cocoylamidopropylbetaine, and mixtures thereof.

More preferentially, the amphoteric or zwitterionic surfactant(s) are chosen from cocoylamidopropylbetaine and cocoylbetaine.

The non-ionic surfactants other than the non-ionic surfactants of formula (I) that may be present in the composition of the invention can be chosen in particular from polyoxyalkylenated fatty alcohols, alkyl polyglucosides (APGs), oxyalkylenated glycerol esters, oxyalkylenated fatty acid esters of sorbitan, polyoxyalkylenated (in particular polyoxyethylenated and/or polyoxypropylenated) fatty acid esters optionally in combination with a fatty acid ester of glycerol, such as the PEG-100 stearate/glyceryl stearate mixture sold for example by the company ICI under the name Arlacel 165, oxyalkylenated sugar esters, and mixtures thereof.

Unless otherwise mentioned, the term “fatty” compound (for example a fatty acid, a fatty alcohol) is intended to denote, for these non-ionic surfactants other than the non-ionic surfactants of formula (I) a compound comprising in its main chain at least one saturated or unsaturated alkyl chain comprising at least six carbon atoms, preferably from 8 to 30 carbon atoms, and even better still from 10 to 22 carbon atoms.

As fatty alcohols, use is preferably made of those chosen from alcohols, alpha-diols and (C₁-20)alkylphenols, other than the non-ionic surfactants of formula (I), comprising in their main chain at least one saturated or unsaturated, more preferentially saturated, alkyl chain comprising at least six carbon atoms; these fatty alcohols being polyoxyalkylenated, such as polyethoxylated and/or polypropoxylated and/or polyglycerolated, the number of ethylene oxide and/or propylene oxide groups possibly ranging from 1 to 100, and the number of glycerol groups possibly ranging from 2 to 30.

Among these polyoxyalkylenated, in particular polyoxyethylenated, fatty alcohols, mention may more particularly be made of oleth-3, oleth-5, laureth-4, ceteareth-10, ceteareth-20, and mixtures thereof.

Use is preferably made, as alkyl polyglucosides, of those containing an alkyl group comprising from 6 to 30 carbon atoms and preferably from 8 to 16 carbon atoms and containing a glucoside group preferably comprising from 1.2 to 3 glucoside units. The alkylpolyglucosides may be chosen, for example, from decylglucoside (alkyl-C9/C11-polyglucoside (1.4)), for instance the product sold under the name Mydol 10® by the company Kao Chemicals or the product sold under the name Plantacare 2000 UP® by the company Cognis; caprylyl/capryl glucoside, for instance the product sold under the name Plantacare KE 3711® by the company Cognis; laurylglucoside, for instance the product sold under the name Plantacare 1200 UP® by the company Cognis; cocoyl glucoside, for instance the product sold under the name Plantacare 818 UP® by the company Cognis; caprylylglucoside, for instance the product sold under the name Plantacare 810 UP® by the company Cognis; and mixtures thereof.

The oxyalkylenated glycerol esters are especially polyoxyethylenated derivatives of esters of glycerol and of a fatty acid and of the hydrogenated derivatives thereof. These oxyalkylenated glycerol esters can be chosen, for example, from glyceryl esters of fatty acids which are hydrogenated and oxyethylenated, such as PEG-200 hydrogenated glyceryl palmate, sold under the name Rewoderm LI-S 80 by the company Goldschmidt; oxyethylenated glyceryl cocoates, such as PEG-7 glyceryl cocoate, sold under the name Tegosoft GC by the company Goldschmidt, and PEG-30 glyceryl cocoate, sold under the name Rewoderm LI-63 by the company Goldschmidt; oxyethylenated glyceryl stearates; and mixtures thereof.

The oxyalkylenated sugar esters are especially polyethylene glycol ethers of fatty acid and sugar esters. These oxyalkylenated sugar esters may be chosen, for example, from oxyethylenated glucose esters, such as PEG-120 methyl glucose dioleate, sold under the name Glucamate DOE 120 by the company Amerchol.

The surfactant(s) can also be chosen from silicone surfactants, for instance dimethicone copolyols, such as the mixture of cyclomethicone and of dimethicone copolyol, sold under the name DC 5225 C® by the company Dow Corning, and alkyldimethicone copolyols such as laurylmethicone copolyol sold under the name Dow Corning 5200 Formulation Aid by the company Dow Corning; cetyldimethicone copolyol, such as the product sold under the name Abil EM 90R® by the company Goldschmidt, and the mixture of cetyldimethicone copolyol, of polyglyceryl isostearate (4 mol) and of hexyl laurate, sold under the name Abil WE O9® by the company Goldschmidt. One or more coemulsifiers, which may be chosen advantageously from the group comprising polyol alkyl esters, may also be added thereto.

Mention may also be made of non-silicone emulsifying surfactants, in particular alkyl esters or ethers of a polyol. As alkyl esters of a polyol, mention may in particular be made of polyethylene glycol esters, for instance PEG-30 dipolyhydroxystearate, such as the product sold under the name Arlacel P135® by the company ICI.

According to one preferred embodiment of the invention, the composition according to the invention also comprises one or more additional surfactants chosen from non-ionic surfactants other than the non-ionic surfactants of formula (I) as defined above, anionic surfactants, amphoteric or zwitterionic surfactants, cationic surfactants, as described above, and mixtures thereof.

More preferentially, the composition according to the invention comprises one or more additional surfactants chosen from non-ionic surfactants other than the non-ionic surfactants of formula (I) as defined above, cationic surfactants, and mixtures thereof; even more preferentially from polyoxyethylenated alcohols other than the non-ionic surfactants of formula (I) as defined above, the cationic surfactants of formula (XI) as defined above, and mixtures thereof.

Preferably, the total content of the additional surfactant(s) optionally present in the composition according to the invention is between 0.01% and 20% by weight, more preferentially between 0.05% and 15% by weight, even more preferentially between 0.1% and 10% by weight, even better still between 0.5% and 5% by weight, relative to the total weight of the composition.

The Additives

The composition according to the invention may optionally also comprise one or more additives, such as natural or synthetic thickeners or viscosity regulators other than the fixing polymers described above; vitamins or provitamins; amphoteric or anionic polymers other than the fixing polymers described above; preservatives; dyes; fragrances.

Those skilled in the art will take care to select the optional additives and the amount thereof such that they do not harm the properties of the composition of the present invention.

These additives are generally present in the composition according to the invention in an amount ranging from 0 to 20% by weight relative to the total weight of the composition.

The pH of the composition generally ranges from 3 to 9, preferably from 3 to 7.5, preferentially from 3.5 to 7 and even better still from 4 to 6.8.

The pH of the composition may be adjusted to the desired value by means of basifying agents or acidifying agents that are customarily used. Among the basifying agents, examples that may be mentioned include aqueous ammonia, alkanolamines, and mineral or organic hydroxides. Among the acidifying agents that may be mentioned, for example, are mineral or organic acids, for instance hydrochloric acid, orthophosphoric acid or sulfuric acid, carboxylic acids, for instance acetic acid, tartaric acid, citric acid and lactic acid, and sulfonic acids.

A subject of the invention is also an aerosol device comprising a composition according to the invention as previously described, a container containing said composition, and a means for spraying said composition.

Preferably, the aerosol device according to the invention makes it possible to spray said composition in mousse form.

The composition according to the invention is advantageously packaged under pressure, in an aerosol device, for example a monobloc device, which comprises a spraying means and a container.

The spraying means is generally formed from a dispensing valve controlled by a dispensing head, which itself comprises a nozzle via which the composition of the invention is sprayed, preferably in mousse form. For example, a Valve Precision P14105 or GI 2×0.51 mm, and a diffuser DMPR229 can be used.

The container containing the pressurized composition may be opaque or transparent. It may be made of glass, polymer or metal, and may optionally be coated with a protective varnish coat.

Preferably, the container of said aerosol device is transparent, such that the composition according to the invention is visible to the naked eye through said container.

Finally, a subject of the invention is a process for styling, that is to say of shaping and/or of fixing, keratin fibres, in particular human keratin fibres such as the hair, comprising the application to said fibres of the composition as previously described, this application being optionally followed by rinsing after an optional leave-on-time.

Preferably, the application of the composition according to the invention is not followed by rinsing.

In a first embodiment of the process according to the invention, the composition is applied to wet hair.

In a second embodiment of the process according to the invention, the composition is applied to dry hair.

The examples that follow serve to illustrate the invention without, however, being limiting in nature.

### EXAMPLES

The following nanoemulsions of oil-in-water type A, B, C and D were prepared from the ingredients detailed in the table below, in which all the amounts are indicated as weight percentages of active material (AM) relative to the total weight of the composition.

Composition A:

| | Content (weight |
|---|---|
| Ingredients | % of AM) |
| Oleth-10 | 14.25 |
| Coconut oil | 8.08 |
| Liquid paraffin | 8.08 |
| Isobutane/propane/butane mixture | 5 |
| Water | qs 100 |

Composition B:

| | Content (weight |
|---|---|
| Ingredients | % of AM) |
| Oleth-10 | 14.25 |
| Coconut oil | 8.08 |
| Liquid paraffin | 8.08 |
| Vinylpyrrolidone/vinyl acetate | 0.48 |
| copolymer | |
| Isobutane/propane/butane mixture | 5 |
| Water | qs 100 |

Composition C:

| | Content (weight |
|---|---|
| Ingredients | % of AM) |
| Oleth-10 | 14.25 |
| Coconut oil | 8.08 |
| 2-Ethylhexyl stearate | 8.08 |
| Isobutane/propane/butane mixture | 5 |
| Water | qs 100 |

Composition D:

| | Content (weight |
|---|---|
| Ingredients | % of AM) |
| Oleth-10 | 14.25 |
| Coconut oil | 8.08 |
| 2-Ethylhexyl stearate | 8.08 |
| Vinylpyrrolidone/vinyl acetate | 0.48 |
| copolymer | |
| Sodium chloride | 0.04 |
| Isobutane/propane/butane mixture | 5 |
| Water | qs 100 |

Composition E:

| | Content (weight |
|---|---|
| Ingredients | % of AM) |
| Oleth-10 | 3.56 |
| Coconut oil | 2.02 |
| Liquid paraffin | 2.02 |
| Vinylpyrrolidone/vinyl acetate copolymer | 1.9 |
| Cetyltrimethylammonium chloride | 0.9 |
| Oleocetyldimethylhydroxyethylammonium | 0.57 |
| chloride | |
| Quaternium-80 | 0.48 |
| Glycerin | 4.75 |
| Propylene glycol | 0.47 |
| Isobutane/propane/butane mixture | 5 |
| Water | qs 100 |

Composition F:

| | Content (weight % |
|---|---|
| Ingredients | of AM) |
| Oleth-10 | 9.98 |
| Coconut oil | 5.65 |
| Liquid paraffin | 5.65 |
| Isobutane/propane/butane mixture | 5 |
| Water | qs 100 |

The compositions above have been prepared according to a phase inversion (PIT) process comprising the following steps:
the non-ionic surfactant of formula (I) and the water are mixed and heated to 50° C.; then
the fatty substances (ii), having a melting point less than or equal to 35° C. at atmospheric pressure, are added to the mixture and heated to 80° C.; then
the solution obtained is cooled to 25° C. by thermal shock; then
the other ingredients are added to the solution, and optionally the solution can be diluted up to 25% for example.

At this stage of the process, the oil particles of the prepared solutions have a number-average size of about 11.5 nm, and the turbidity of the prepared solutions is about 70 NTU units, measured at ambient temperature (25° C.) and atmospheric pressure.

The number-average size of the particles (or oil drops) has been determined according to the known Dynamic Light Scattering (DLS) method with the Zetasizer Nano ZS granulometer from Malvern Instrument. This granulometer is equipped with a standard laser (Helium-Neon) of 4 mW of power at a wavelength λ of 633 nm.

For this purpose, the prepared solutions have been previously diluted with distilled water with a dilution ratio of 1:100, and the temperature of each sample has been regulated at 25° C.

The NNLS (Non-Negatively Least Squares) correlation function is used to analyse the data.

The turbidity measurements of the prepared solutions have been carried out with a turbidimeter, model HI 88713-ISO from the company Hanna Instruments.

Then, the mixture of C₃-C₅ alkanes is emulsified into the prepared solutions, to obtain the compositions according to the invention. The mixture of C₃-C₅ alkanes is entirely dispersed in the composition and does not form a separate layer.

The nanoemulsions of oil-in-water type A, B, C, D and E above are clear, with a single-phase appearance.

The single-phase appearance and the clarity of the composition are stable over time. In particular, after 2 months of storage at ambient temperature (25° C.), the appearance of the composition has not changed.

Each composition A, B, C, D and E above was packaged in an aerosol device comprising a container containing said composition, and a means for spraying said composition (Equipment: a Valve Precision P14105 or GI 2×0.51 mm, and a diffuser DMPR229).

On spraying, a firm, creamy mousse is obtained, which spreads easily on the head of hair and gives good styling and cosmetic properties, in particular with long-lasting fixing and a pleasant feel. The hair is soft, and has volume. The hairstyle obtained is natural, with no helmet effect.

1. A cosmetic composition in the form of a nanoemulsion of oil-in-water type, comprising:
at least one non-ionic surfactant of formula (I):
R—O—(CH₂—CH₂—O)ₙ—H (I)
in which:
R is a linear or branched C₈-C₄₀ alkenyl radical,
n is an integer ranging from 6 to 20;
(ii) one or more fatty substances having a melting point less than or equal to 35° C. at atmospheric pressure;
(iii) one or more propellants; and
(iv) water.

2. The composition according to claim 1, wherein R represents a linear or branched C₁₆-C₂₀ alkenyl radical; and/or n represents an integer ranging from 8 to 12.

3. The composition according to claim 1, wherein at least one non-ionic surfactant of formula (I) is chosen from oleyl alcohol comprising 8 mol of ethylene oxide, oleyl alcohol comprising 10 mol of ethylene oxide, oleyl alcohol comprising 12 mol of ethylene oxide.

4. The composition according to claim 1, wherein the total content of the non-ionic surfactant(s) of formula (I) is between 0.1% and 15% by weight relative to the total weight of the composition.

5. The composition according to claim 1, wherein the fatty substance(s) (ii) are chosen from fatty substances that are liquid at 28° C. and at atmospheric pressure.

6. The composition according to claim 1, wherein it comprises at least two fatty substances having a melting point less than or equal to 35° C. at atmospheric pressure, one of said fatty substances being chosen from hydrocarbons, and the other of said fatty substances from vegetable oils.

7. The composition according to claim 1, wherein the weight ratio of the content of non-ionic surfactant(s) of formula (I) to the content of the fatty substance(s) (ii), is inclusively between 0.05 and 20.

8. The composition according to claim 1, wherein the propellant(s) are chosen from volatile hydrocarbons which are optionally halogenated, dimethyl ether, and mixtures thereof.

9. The composition according to claim 1, wherein the total content of the propellant(s) is between 0.1% and 20% by weight relative to the total weight of the composition.

10. The composition according to claim 1, wherein that the propellant(s) used are entirely dispersed in the composition before the first use of the composition.

11. Composition according to claim 1, wherein it also comprises one or more fixing polymers chosen from non-ionic fixing polymers, amphoteric fixing polymers, anionic fixing polymers, cationic fixing polymers, and mixtures thereof; preferably chosen from non-ionic fixing polymers, anionic fixing polymers, and mixtures thereof; more preferentially chosen from non-ionic fixing polymers; even more preferentially chosen from vinyllactam homopolymers such as vinylpyrrolidone homopolymers and polyvinylcaprolactam, and vinyllactam copolymers, such as poly(vinylpyrrolidone/vinyllactam) copolymers and poly(vinylpyrrolidone/vinyl acetate) copolymers, and mixtures thereof; preferably in a total amount ranging from 0.1% to 20% by weight, more preferentially from 0.2% to 15% by weight, 0.5% to 10% by weight, better still from 1% to 5% by weight, relative to the total weight of the composition.

12. An aerosol device comprising a composition as defined according to claim 1, a container containing said composition, and a means for spraying said composition.

13. The aerosol device according to claim 12, which makes it possible to spray said composition in the form of a mousse.

14. The aerosol device according to claim 12, wherein the container is transparent.

15. A process for styling keratin fibres comprising the application to said fibres of the composition as defined in claim 1, this application being optionally followed by rinsing after an optional leave-on time.

16. The composition according to claim 2, wherein at least one non-ionic surfactant of formula (I) is chosen from oleyl alcohol comprising 8 mol of ethylene oxide, oleyl alcohol comprising 10 mol of ethylene oxide, oleyl alcohol comprising 12 mol of ethylene oxide.

17. The composition according to claim 2, wherein the total content of the non-ionic surfactant(s) of formula (I) is between 0.1% and 15% by weight relative to the total weight of the composition.

18. The composition according to claim 3, wherein the total content of the non-ionic surfactant(s) of formula (I) is between 0.1% and 15% by weight relative to the total weight of the composition.

19. The composition according to claim 2, wherein the fatty substance(s) (ii) are chosen from fatty substances that are liquid at 28° C. and at atmospheric pressure.

20. The composition according to claim 3, wherein the fatty substance(s) (ii) are chosen from fatty substances that are liquid at 28° C. and at atmospheric pressure.
